(19) United States
(12) Patent Application Publication
(10) Pub. No.: US 2026/0130581 A1
(43) Pub. Date: May 14, 2026

(54) MEDICAL DEVICES, SYSTEMS, AND METHODS FOR ADJUSTING FLUID FLOW

(71) Applicant: Boston Scientific Scimed, Inc., Maple Grove, MN (US)

(72) Inventors: John SUBASIC, Sutton, MA (US); Amy BRADLEY, Westford, MA (US)

(73) Assignee: Boston Scientific Scimed, Inc., Maple Grove, MN (US)

(21) Appl. No.: 19/383,931

(22) Filed: Nov. 10, 2025

Related U.S. Application Data
(60) Provisional application No. 63/719,745, filed on Nov. 13, 2024.

(51) Int. Cl. A61B 1/015; A61B 1/00; A61B 1/018; A61B 1/04; A61B 1/12

(52) U.S. Cl. CPC A61B 1/015; A61B 1/00068; A61B 1/00128; A61B 1/018; A61B 1/04; A61B 1/126

(57) ABSTRACT

Medical systems and devices configured to adjust fluid flow are described. The medical system may include a medical device having a handle coupled to a shaft that includes at least one working channel. The handle may include a port in communication with the at least one working channel and configured to connect to a source of fluid or vacuum. The medical system also may include a control mechanism coupled to or integrated into the handle. The control mechanism may include a grippable portion configured to adjust fluid flow through the at least one working channel of the medical device when a user squeezes the grippable portion or moves the grippable portion relative to the handle.

### CROSS-REFERENCE TO RELATED APPLICATIONS

This application claims the benefit of priority to U.S. Provisional Application No. 63/719,745, filed on Nov. 13, 2024, which is incorporated by reference herein in its entirety.

### TECHNICAL FIELD

Aspects of this disclosure generally relate to medical devices, systems, and related methods for adjusting fluid flow during medical procedures. In particular, aspects of the disclosure relate to medical systems that include mechanisms for adjusting fluid flow helpful for clearing working channels.

### BACKGROUND

Medical devices such as scopes and sheaths are often inserted into the body of a patient to perform a therapeutic and/or diagnostic procedure inside the body. During medical procedures, such as endoscopic or ureteroscopic procedures, a medical professional may use an aspiration sheath or scope coupled to a vacuum or fluid source to provide suction or irrigation. When removing material from the body, the working channel of a device may become blocked and inhibit the ability to continue with a procedure. Clearing such blockages by removing the device from the body or disassembling and reassembling suction or irrigation connections is cumbersome and can unnecessarily prolong procedures.

### SUMMARY

The disclosure includes medical systems and related medical devices and methods to adjust fluid flow. For example, the medical system may comprise a medical device including a handle coupled to a shaft extending to a distal end of the medical device, e.g., the shaft including at least one working channel extending from a proximal end of the shaft to a distal end of the shaft. The handle may include a port in communication with the at least one working channel and configured to connect to a source of fluid or vacuum. The medical system also includes a control mechanism coupled to or integrated into the handle. The control mechanism may include a grippable portion configured to adjust fluid flow through the at least one working channel when a user squeezes the grippable portion or moves the grippable portion relative to the handle. For example, the user may move the grippable portion distally relative to the handle to adjust fluid flow.

According to some aspects, the grippable portion of the control mechanism may include a bulb in fluid communication with the at least one working channel of the medical device, e.g., through the port of the medical device. In some examples, the port may be a three-way port including a first arm in fluid communication with the control mechanism and a second arm configured to receive a medical instrument, such as a scope or a laser. The bulb may include an inlet fluidly coupled to the port via a first tubing and an outlet fluidly coupled to a second tubing. The outlet may be adjacent to the inlet and the grippable portion may include the second tubing. In some aspects, the outlet may be opposite the inlet. Furthermore, in some examples, the bulb may be compressible and comprise an elastic polymeric material. Optionally, the control mechanism may include a one-way valve in fluid communication with the bulb.

In some aspects, the control mechanism may include a bellows. For example, the bellows may be compressible and expandable and/or may comprise a flexible polymer. The grippable portion and the bellows of the control mechanism may be movable relative to the shaft along a longitudinal axis of the medical device. In some aspects, the grippable portion may include a ring configured to receive a thumb of the user's hand. The grippable portion, e.g., the ring, may be rotatable relative to the handle of the medical device. Additionally, or alternatively, in some examples, the control mechanism may include a lock configured to maintain a position of the grippable portion relative to the handle.

In some aspects, the handle may include a proximal opening with a seal. The medical system may further comprise a scope, and a shaft of the scope may extend through the seal and extend through the at least one working channel.

The disclosure also includes a medical system comprising a medical device that includes a handle coupled to a shaft extending to a distal end of the medical device. The shaft may include at least one working channel extending from a proximal end of the shaft to a distal end of the shaft. The handle may include a port in communication with the at least one working channel and configured to connect to a source of fluid or vacuum. The medical system also includes a control mechanism coupled to the handle of the medical device. The control mechanism may include a grippable portion including a bulb configured to adjust fluid flow through the at least one working channel when a user squeezes the bulb. The bulb may be in fluid communication with the at least one working channel through the port. According to some aspects, the bulb may be compressible and comprise an elastic polymeric material. The control mechanism may include an inlet fluidly coupling the bulb to the port via a first tubing and an outlet fluidly coupling the bulb to a second tubing. The outlet may be opposite the inlet.

The disclosure also includes a medical system comprising a medical device including a handle coupled to a shaft extending to a distal end of the medical device, the shaft including at least one working channel extending from a proximal end of the shaft to a distal end of the shaft. The handle may include a port in communication with the at least one working channel and configured to connect to a source of fluid or vacuum. The medical system also includes a control mechanism integrated into the handle. The control mechanism may comprise a grippable portion, and a bellows configured to adjust fluid flow through the at least one working channel when a user moves the control mechanism along a longitudinal axis of the medical device. According to some aspects, the control mechanism may include a lock configured to maintain a position of the grippable portion relative to the handle.

### BRIEF DESCRIPTION OF THE FIGURES

The accompanying drawings, which are incorporated in and constitute a part of this application, illustrate exemplary aspects that, together with the written description, explain the principles of this disclosure. The figures depict exemplary aspects according to this disclosure, as follows:

FIG. 1 shows a side view of an exemplary medical system with a control mechanism used for adjusting fluid flow, according to aspects of this disclosure;

FIG. 2 shows a side view of another exemplary medical system with a control mechanism, according to aspects of this disclosure; and

FIGS. 3A-3B show a side view of another exemplary medical system with a control mechanism that includes a bellows, according to aspects of this disclosure.

### DETAILED DESCRIPTION

Particular aspects of the present disclosure are described in greater detail below. The terms and definitions provided herein control, if in conflict with terms and/or definitions incorporated by reference. The terms “proximal” and “distal” are used herein to refer to the relative positions of the components of exemplary medical devices. As used herein, “proximal” refers to a position relatively closer to the exterior of the body or closer to a user or an operator (e.g., a physician or other medical professional) using the medical device. In contrast, “distal” refers to a position relatively further away from the operator using the medical device, or closer to the interior of the body.

Both the foregoing general description and the following detailed description are exemplary and explanatory only and are not restrictive of the features, as claimed. As used herein, the terms “comprises,” “comprising,” “having,” “including,” or other variations thereof, are intended to cover a non-exclusive inclusion such that a process, method, article, or apparatus that comprises a list of elements does not include only those elements but may include other elements not expressly listed or inherent to such a process, method, article, or apparatus. In this disclosure, relative terms, such as, for example, “about,” “substantially,” “generally,” and “approximately” are used to indicate a possible variation of ±10% in a stated value or characteristic.

Although aspiration sheaths and scopes such as ureteroscopes are referenced herein for illustration purposes, it will be appreciated that the disclosure encompasses any suitable medical device configured to allow an operator to access and view internal body anatomy of a subject and/or to deliver suction, irrigation, and/or medical instruments such as, for example, biopsy forceps, graspers, baskets, snares, probes, scissors, capture/retrieval devices, lasers, and other tools, into the subject's body. The medical devices herein may be inserted into a variety of body lumens and/or cavities, such as, for example, the urinary tract or gastrointestinal tract. It will be appreciated that, unless otherwise specified, bronchoscopes, duodenoscopes, endoscopes, gastroscopes, endoscopic ultrasonography (“EUS”) scopes, colonoscopes, ureteroscopes, bronchoscopes, laparoscopes, cystoscopes, aspiration sheaths, catheters, or any other suitable delivery device may be used in connection with the features described herein.

Aspects of this disclosure are described with reference to exemplary medical systems that facilitate clearing a blockage of a working channel of a medical device, e.g., via manual adjustment of fluid flow through the working channel. For example, a user (e.g., a medical professional) may use features of the medical systems herein to clear obstructions that may occur in the working channel during medical procedures involving irrigation and/or suction.

According to some aspects, the medical system may include a medical device with a handle attached to a shaft that extends to a distal end of the medical device. The shaft may include at least one working channel extending along its length from a proximal end to the distal end. The handle may include a port that may be fluidly coupled to the working channel and may be coupled to a fluid or vacuum source that provides fluid flow or negative pressure during a medical procedure. Additionally, the medical system may include a control mechanism with a grippable portion that the user may actuate, e.g., temporarily, to manually adjust fluid flow in the working channel of the medical device, which may help clear blockages in the working channel. The adjustment may include a temporary change in pressure and/or delivery of a bolus of fluid through the channel. Once the obstruction is cleared, the user may return to conditions before deploying the control mechanism.

Reference will now be made in detail to examples to help illustrate aspects of the present disclosure through the accompanying drawings. Wherever possible, the same reference numbers will be used throughout the drawings to refer to the same or like parts.

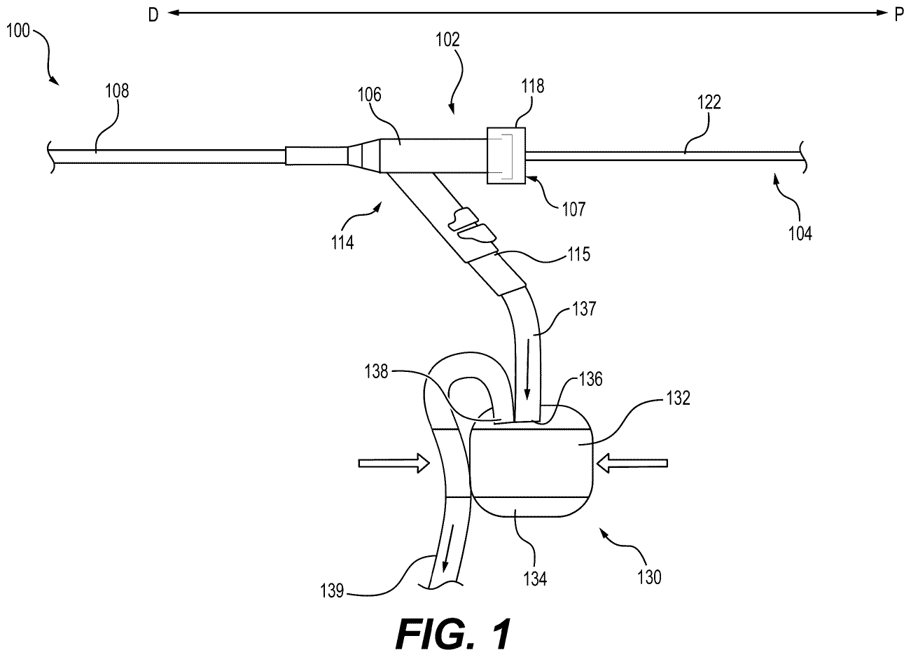

FIG. 1 depicts an exemplary medical system 100. Medical system 100 includes a medical device 102, which may be an aspiration sheath as shown in this example, and a control mechanism 130. Optionally, medical system 100 may include a medical instrument such as a scope 104 received by medical device 102. Scope 104 may be any suitable medical scope configured to allow a user to access, view internal areas of a subject's body, and perform medical diagnoses and/or treatments on a subject. For example, scope 104 may be a ureteroscope, an endoscope, a hysteroscope, a bronchoscope, a cystoscope, or other scopes or similar medical devices.

Medical device 102 may include a handle 106 and a shaft 108. Shaft 108 may include a working channel in fluid communication with a port 114 of handle 106, shaft 108 extending distally from handle 106. Handle 106 may include a proximal opening 107 in fluid communication with the working channel and the port 114, proximal opening 107 configured to receive scope 104, e.g., a shaft 122 of scope 104. Shaft 122 may be slidable relative to medical device 102, such that scope shaft 122 passes through the working channel of shaft 108. Further, for example, the proximal opening 107 of handle 106 may include a seal 118 configured to form a fluid-tight seal against shaft 122 of scope 104. Shaft 122 may be flexible and configured to bend or deflect during navigation toward, and away from, a target site within a body of a patient. Shaft 122 may also define one or more working channels, e.g., to transport medical tools, supply fluid, provide suction, etc., during a medical procedure.

Still referring to FIG. 1 and as noted above, medical system 100 includes control mechanism 130 coupled to port 114 of handle 106 of medical device 102. Control mechanism 130 includes a grippable portion 132 that may include a bulb 134 in fluid communication with the working channel of shaft 108. For example, bulb 134 may be in fluid communication with the working channel of shaft 108 via port 114, e.g., through flexible tubing. In some aspects, bulb 134 may comprise a compressible, elastic polymeric material, configured to allow repeated cycles of compression and decompression. The elastic, polymeric material may help bulb 134 return to its original shape after a user has squeezed and released grippable portion 132. In some aspects, handle 106 of medical device 102 and/or control mechanism 130 may include a one-way valve in fluid communication with the working channel of shaft 108 and bulb 134. The one-way valve may help facilitate unidirectional fluid flow.

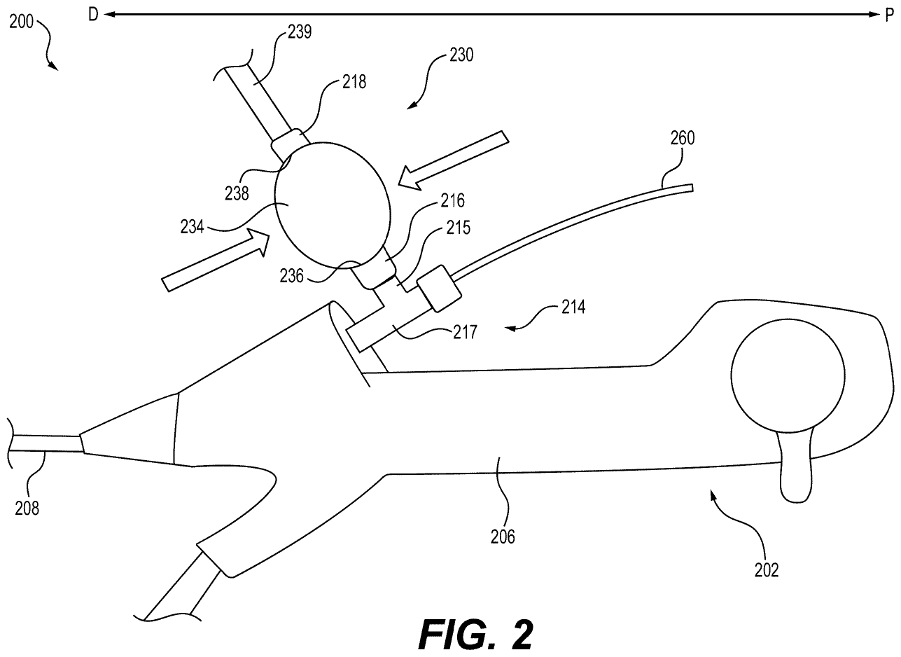

Bulb 134 may include an inlet 136 and an outlet 138. In some aspects, outlet 138 may be located adjacent to inlet 136 as illustrated in FIG. 1. Alternatively, outlet 138 may be positioned opposite inlet 136 (see, e.g., FIG. 2 discussed below). Bulb 134 may be fluidly coupled to inlet 136 and port 114 via a first tubing 137, thereby fluidly coupling bulb 134 to the working channel of shaft 108 through port 114. In addition, a second tubing 139 may fluidly couple outlet 138 to a source of suction, optionally via a fluid management system. Grippable portion 132 may include at least a portion of second tubing 139, such that second tubing 139 may be positioned adjacent to and/or in contact with a surface of bulb 134.

During use, vacuum pressure may be applied to the working channel of medical device 102 via a source of suction, wherein the suction line includes control mechanism 130. In some aspects, when a user squeezes grippable portion 132, the user may compress both bulb 134 and second tubing 139, thereby temporarily interrupting the flow of suction pressure through second tubing 139. As a result, fluid (e.g., air, liquid, etc. ,) may apply pressure back through bulb 134 and into the working channel of shaft 108 via port 114, creating a reverse fluid flow. The user may release grippable portion 132 to reestablish suction pressure through the working channel via bulb 134 of control mechanism 130.

During an exemplary medical procedure, a distal portion of shaft 108 of medical device 102 may be at least partially inserted into an orifice of the subject proximate a target site of a subject and positioned within the urinary or gastrointestinal system. Shaft 122 of scope 104 may be inserted through proximal opening 107 and advanced through shaft 108 of medical device 102. During the procedure, a user may apply suction through the working channel via port 114 of medical device 102 to remove fluids, tissue, and/or other materials from a target site of a subject. If the working channel becomes clogged with material, the user may squeeze grippable portion 132 of control mechanism 130 as mentioned above to temporarily cut off suction and to reverse fluid flow through the working channel of shaft 108. As a result, fluid within bulb 134 may be reintroduced into the working channel of shaft 108 via port 114. Pressure applied through the reverse fluid flow may help to dislodge obstructions such as stone fragments, tissue, or debris that may block fluid flow within the working channel of shaft 108. Once the user releases bulb 134, bulb 134 returns to its predefined shape and suction pressure is restored through second tubing 139.

FIG. 2 depicts another exemplary medical system 200 according to aspects of the present disclosure. Medical system 200 may include any of the features of medical system 100, except as described herein. Medical system 200 includes a medical device 202 that includes a handle 206 and a shaft 208, and a control mechanism 230 coupled to medical device 202. Medical device 202 and control mechanism 230 may include any of the features of medical device 102 and control mechanism 130, respectively. Medical device 202 may be a scope, e.g., configured to allow a user to access, view internal areas of a subject's body, and perform medical diagnoses and/or treatments on a subject. For example, medical device 202 may be a ureteroscope, an endoscope, a hysteroscope, a bronchoscope, a cystoscope, or other scopes or similar medical devices. In some examples, medical device 202 may be configured to receive a medical instrument via a port 214 of handle 206 (e.g., rather than via a proximal opening of a handle as illustrated in FIG. 1) for passage through shaft 108 of medical device 202 to reach a target site of a subject.

Shaft 208 may include a working channel in fluid communication with port 214 of handle 206, shaft 208 extending distally from handle 206. In this example, port 214 is shown as a three-way port, e.g., including a first arm 215 and a second arm 217. For example, first arm 215 may be coupled to control mechanism 230, and second arm 217 may be configured to receive a medical instrument 260. For example, second arm 217 may be sized to permit passage of various medical instruments (e.g., a laser fiber or other instrument) therethrough, for passage through the working channel of shaft 208. In some examples, handle 206 may include separate ports in fluid communication with separate working channels of shaft 208. For example, control mechanism 230 may be coupled to port 214 in communication with a first working channel and handle 206 may include an additional port configured to receive a medical instrument 260 for passage through a second working channel of shaft 208.

Control mechanism 230 may include a bulb 234 having an inlet 238 and an outlet 236. While the following discussion refers to the control mechanism 230 in the context of irrigation (e.g., providing fluid to the working channel of shaft 208), it is understood that these terms are for convenience only. For example, control mechanism 230 may be used in connection with suction, wherein fluid is removed from the working channel (e.g., a direction of flow from outlet 236 to inlet 238). Bulb 234 may be similar to bulb 134, e.g., comprising a compressible, elastic polymeric material. Thus, bulb 234 may be compressible and may return to its original shape after a user has squeezed and released a grippable portion (e.g., a central portion) of bulb 234.

Inlet 238 and an outlet 236 may be positioned opposite each other. In some aspects, a first tubing 216, optionally including a valve, may couple outlet 236 to port 214. In addition, a second tubing 239 may fluidly couple inlet 238 to a source of fluid (or suction), optionally via a fluid management system. Second tubing 239 may include a valve.

During an exemplary medical procedure, a distal portion of shaft 208 of medical device 202 may be positioned proximate a target site of a subject, e.g., within the urinary or gastrointestinal system. During the procedure, a user may supply fluid through the working channel of shaft 208 to dislodge or clear blood, tissue, and/or other materials from the target site. If the working channel becomes clogged with material, the user may temporarily squeeze bulb 234 of control mechanism 230 to provide a bolus of fluid (e.g., force a volume of fluid retained within bulb 234) through the working channel of shaft 208. As a result, the bolus of fluid may help to dislodge obstructions such as stone fragments, tissue, or debris that may be blocking fluid flow within the working channel of shaft 208. Once the user releases bulb 234, bulb 234 returns to its predefined shape and steady state irrigation may be restored through second tubing 239.

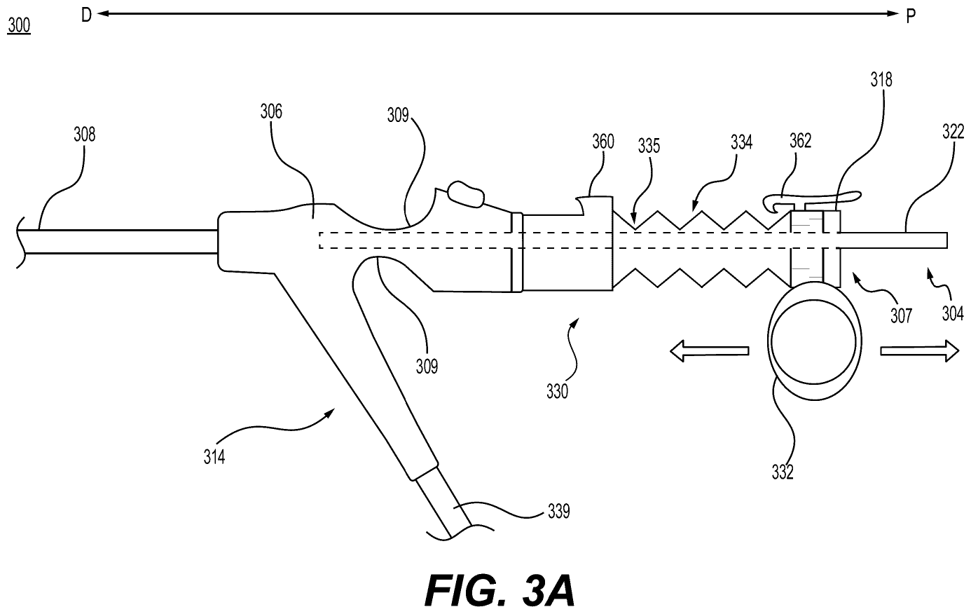

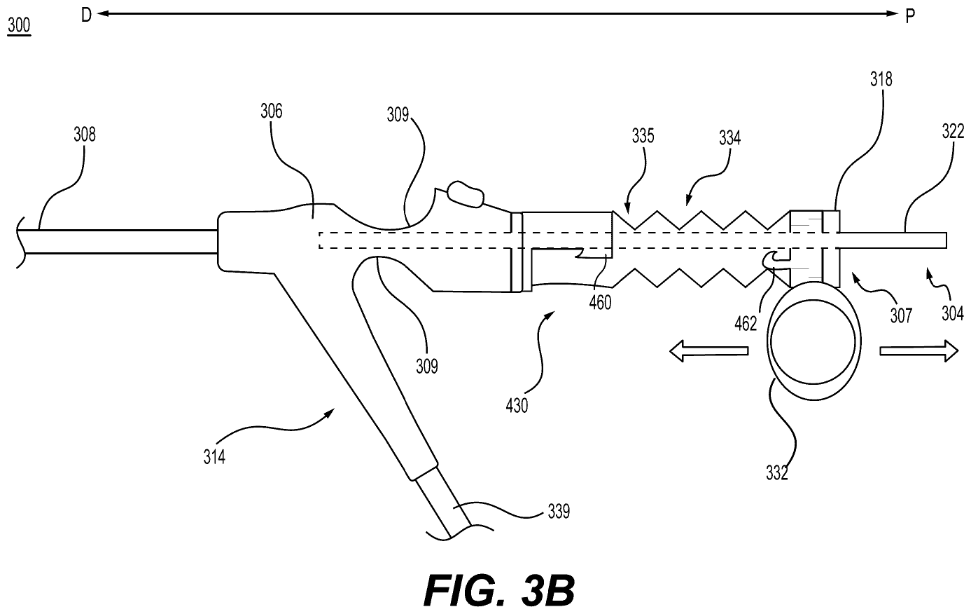

FIGS. 3A and 3B depict another exemplary medical system 300 according to aspects of the present disclosure. FIGS. 3A and 3B differ with respect to an optional lock, discussed further below. Medical system 300 may include any of the features of medical system 100 and/or 200, except as described herein. Medical system 300 includes a medical device 302 and a control mechanism 330. Medical device 302 may be an aspiration sheath similar to medical device 102 with different handle features. Optionally, medical system 300 may include a medical instrument such as a scope 304 received by medical device 302. Scope 304 may be any suitable scope, e.g., configured to allow a user to access, view internal areas of a subject's body, and perform medical diagnoses and/or treatments on a subject.

Medical device 302 includes a handle 306 and a shaft 308. Optionally, handle 306 may include ergonomic features to assist with handling of medical device 302 in use. As shown, for example, handle 306 includes curved portions 309 to receive fingers (e.g., an index finger and a middle finger) of the user's hand as the thumb of the hand moves a control mechanism 330, described further below. Thus, for example, curved portions 309 are shown on opposite sides of handle 306. In other aspects, handle 306 does not include such curved portions 309, more similar to handle 106 of medical device 102.

Shaft 308 may include a working channel in fluid communication with a port 314 of handle 306, shaft extending distally from handle 306. Port 314 may be coupled to a source of suction or fluid, e.g., via tubing 339, optionally via a fluid management system. Vacuum pressure or fluid may be provided to the working channel of medical device 302 via port 314. In some aspects, handle 306 of medical device 302 may include a one-way valve to facilitate unidirectional fluid flow through the working channel. Handle 306 may include a proximal opening 307 configured to receive a shaft 322 of scope 304 (e.g., similar to shaft 122 of scope 104). In some aspects, handle 306 includes a seal 318 around proximal opening 307 to form a fluid-tight seal against shaft 322 of scope 304.

Still referring to FIGS. 3A and 3B and as noted above, medical system 300 includes control mechanism 330 coupled to or integrated into handle 306 of medical device 302. In this example, control mechanism is integrated into handle 306. Control mechanism 330 includes a grippable portion 332, illustrated as a ring configured to receive the thumb of a user's hand, and a bellows 334. Control mechanism 330 may be used to temporarily disrupt pressure within medical device 302 to thereby adjust fluid flow through the working channel of shaft 308.

For example, grippable portion 332 and bellows 334 may be movable relative to shaft 308 (and relative to port 314 of handle 306) along the longitudinal axis of medical device 302. In some aspects, bellows 334 may include one or more folds 335 along its length configured to compress and expand volume within handle 306 as grippable portion 332 is moved along the longitudinal axis of medical device 302. Fold(s) 335 may help to reduce resistance and facilitate quick and repeatable movement of control mechanism 330. Bellows 334 may comprise a flexible polymer.

Grippable portion 332 of handle 306 may be at or proximate the proximal end of handle 306, and bellows 334 may be between grippable portion 332 and the remainder of handle 306. In some aspects, handle 306 may be configured to permit rotation of control mechanism 330 (e.g., rotatable of grippable portion 332) relative to shaft 308, helping the user adjust grippable portion 332 to a desired position. For example, bellows 334 may be sufficiently flexible to permit rotation of grippable portion 332 relative to the remainder of handle 306. As mentioned above, grippable portion 332 is illustrated in this example as a ring configured to receive a thumb of the user's hand. Thus, the user may slide the ring or grippable portion 332 longitudinally in a proximal-distal direction to expand or contract bellows 334 and thereby increase or decrease a volume within handle 306.

For example, when grippable portion 332 is moved along the longitudinal axis by a user, bellows 334 may be compressed, forcing fluid (e.g., air or liquid) from bellows 334 into the working channel of shaft 308, or may be expanded, drawing fluid from the working channel. For example, proximal movement of grippable portion 332 to expand bellows 334 may generate a plunger-like effect, which may help dislodge obstructions or prevent the buildup of pressure within the working channel of shaft 308. Similarly, distal movement of grippable portion 332 to contract bellows 334 may force pressure into the working channel to similarly clear obstructions.

Optionally, control mechanism 330 may include a lock to help maintain a position of bellows 334. For example, the lock may secure bellows 334 in a position that prevents compression or expansion, likewise fixing the position of grippable portion 332. The lock may include, for example, one or more engagement features such as clips, connectors, magnets, etc.

For example, FIG. 3A illustrates an exemplary lock that includes clips 360, 362 configured to interlock with each other. Clips 360, 362 may be positioned on opposite sides of bellows 334 and may extend or protrude radially outward relative to the longitudinal axis of handle 306 and relative to bellows 334. As shown in FIG. 3A, clip 360 is near the proximal end of handle 306 and clip 362 is on grippable portion 332. Each clip 360, 362 may include a curved or semi-circular feature configured to engage with the corresponding clip on the opposing side. Clip 360 may have a curved feature projecting distally, aligning with the direction in which bellows 334 is compressed (e.g., distally), while clip 362 may have its curved feature projecting proximally. When grippable portion 332 is moved distally to compress bellows 334, clip 360 may engage with clip 362, forming a lock that secures bellows 334 in the compressed position. To release the lock, the user may move grippable portion 332 proximally, uncoupling clip 362 from clip 360 and allowing bellows 334 to return to its expanded, predefined shape.

FIG. 3B depicts another exemplary lock comprising clips 460, 462, which may be similar to clips 360, 362, except as described herein. As shown in FIG. 3B, clip 460 may be positioned near the proximal end of handle 306, while a complementary clip 462 is positioned on grippable portion 332. Clips 460, 462 may also be positioned on opposite sides of bellows 334 and are configured to interlock. In this example, clips 460, 462 do not extend or protrude radially outward relative to bellows 334. Instead, clips 460, 462 are positioned radially inward towards the longitudinal axis of handle 306. This may provide a smaller profile as compared to the example of FIG. 3A.

During an exemplary medical procedure, a distal portion of shaft 308 of medical device 302 may be at least partially inserted into an orifice of the subject and positioned proximate to a target site of a subject, e.g., within the urinary or gastrointestinal system. Shaft 322 of scope 304 may be inserted through proximal opening 307 and advanced through shaft 308 of medical device 302. During the procedure, a user may apply irrigation or suction through the working channel via port 314 of handle 306 to remove or clear fluids, tissue, and/or other materials from a target site of a subject. If a blockage occurs in the working channel, the user may move grippable portion 332 along the longitudinal axis, e.g., proximally, and/or distally, of medical device 302 to thereby expand or compress bellows 334. The change in volume within medical device 302 due to expansion and/or compression of bellows 334 may adjust pressure and fluid flow within the working channel of shaft 308 to help dislodge obstructions that may be blocking fluid flow within the working channel. By repeating movement of grippable portion 332 along the longitudinal axis of medical device 302, the user may alternate between applying and drawing pressure from the working channel of shaft 308 to clear blockages.

While principles of the disclosure are described herein with reference to illustrative aspects for particular medical uses and procedures, the disclosure is not limited thereto. Those having ordinary skill in the art and access to the teachings provided herein will recognize additional modifications, applications, aspects, and substitution of equivalents all fall in the scope of the aspects described herein. Accordingly, the disclosure is not to be considered as limited by the foregoing description.

What is claimed is:

1. A medical system comprising:
a medical device including a handle coupled to a shaft extending to a distal end of the medical device, the shaft including at least one working channel extending from a proximal end of the shaft to a distal end of the shaft, wherein the handle includes a port in communication with the at least one working channel and configured to connect to a source of fluid or vacuum; and
a control mechanism coupled to or integrated into the handle, the control mechanism including a grippable portion configured to adjust fluid flow through the at least one working channel when a user squeezes the grippable portion or moves the grippable portion distally relative to the handle.

2. The medical system of claim 1, wherein the grippable portion of the control mechanism includes a bulb in fluid communication with the at least one working channel through the port.

3. The medical system of claim 2, wherein the port is a three-way port including a first arm in fluid communication with the control mechanism and a second arm configured to receive a medical instrument.

4. The medical system of claim 2, wherein the bulb includes an inlet fluidly coupled to the port via a first tubing and an outlet fluidly coupled to a second tubing.

5. The medical system of claim 4, wherein the outlet is adjacent to the inlet and the grippable portion includes the second tubing.

6. The medical system of claim 4, wherein the outlet is opposite the inlet.

7. The medical system of claim 2, wherein the bulb is compressible and comprises an elastic polymeric material.

8. The medical system of claim 2, wherein the control mechanism includes a one-way valve in fluid communication with the bulb.

9. The medical system of claim 1, wherein the control mechanism includes a bellows, the grippable portion and the bellows being movable relative to the shaft along a longitudinal axis of the medical device.

10. The medical system of claim 9, wherein the grippable portion includes a ring configured to receive a thumb of the user.

11. The medical system of claim 9, wherein the grippable portion is rotatable relative to the handle.

12. The medical system of claim 9, wherein the bellows comprises a flexible polymer.

13. The medical system of claim 9, wherein the control mechanism includes a lock configured to maintain a position of the grippable portion relative to the handle.

14. The medical system of claim 1, wherein the handle includes a proximal opening with a seal.

15. The medical system of claim 14, further comprising a scope, wherein a shaft of the scope extends through the seal and extends through the at least one working channel.

16. A medical system comprising:
a medical device including a handle coupled to a shaft extending to a distal end of the medical device, the shaft including at least one working channel extending from a proximal end of the shaft to a distal end of the shaft, wherein the handle includes a port in communication with the at least one working channel and configured to connect to a source of fluid or vacuum; and
a control mechanism coupled to the handle, the control mechanism including a grippable portion including a bulb configured to adjust fluid flow through the at least one working channel when a user squeezes the bulb;
wherein the bulb is in fluid communication with the at least one working channel through the port.

17. The medical system of claim 16, wherein the control mechanism includes an inlet fluidly coupling the bulb to the port via a first tubing and an outlet fluidly coupling the bulb to a second tubing, and wherein the outlet is opposite the inlet.

18. The medical system of claim 16, wherein the bulb is compressible and comprises an elastic polymeric material.

19. A medical system comprising:
a medical device including a handle coupled to a shaft extending to a distal end of the medical device, the shaft including at least one working channel extending from a proximal end of the shaft to a distal end of the shaft, wherein the handle includes a port in communication with the at least one working channel and configured to connect to a source of fluid or vacuum; and
a control mechanism integrated into the handle, the control mechanism comprising a grippable portion and a bellows configured to adjust fluid flow through the at least one working channel when a user moves the control mechanism along a longitudinal axis of the medical device.

20. The medical system of claim 19, wherein the control mechanism further includes a lock configured to maintain a position of the grippable portion relative to the handle.
